(19) United States
(12) Patent Application Publication
(10) Pub. No.: US 2006/0258727 A1
(43) Pub. Date: Nov. 16, 2006

(54) Olmesartan medoxomil with reduced levels of impurities

(76) Inventors: Lilach Hedvati, Ein-Shemer (IL); Gideon Pilarsky, Holon (IL)

Correspondence Address: KENYON & KENYON LLP, ONE BROADWAY, NEW YORK, NY, 10004, US

(21) Appl. No.: 11/289,242

(22) Filed: Nov. 28, 2005

Related U.S. Application Data
(63) Continuation-in-part of application No. 11/217,473, filed on Sep. 2, 2005.
(60) Provisional application No. 60/640,232, filed on Jan. 3, 2005.

(51) Int. Cl. A61K 31/4184; C07D 403/02

(52) U.S. Cl. 514/381; 548/253

(57) ABSTRACT

The present invention provides the preparation of olmesartan medoxomil containing less than about 0.1% of one or more of the impurities OLM-Me, OLM-Cl, and OLM-eliminate.

This application claims the benefit of U.S. Provisional Patent Application Ser. No. 60/640,232 filed Jan. 3, 2005 and is a continuation-in-part of U.S. patent application Ser. No. 11/217,473 filed Sep. 2, 2005.

### FIELD OF INVENTION

The present invention relates to olmesartan medoxomil with reduced levels of impurities.

### BACKGROUND OF THE INVENTION

The chemical name for olmesartan medoxomil is 4-(1-hydroxy-1-methylethyl)-2-propyl-1-[[2′-(1H-tetrazol-5-yl)[1,1′-biphenyl]-4-yl]methyl]-1H-imidazole-5-carboxylic acid (5-methyl-2-oxo-1,3-dioxol-4-yl)methyl ester (Merck Index 13th ed.).

The chemical structure of olmesartan medoxomil is:

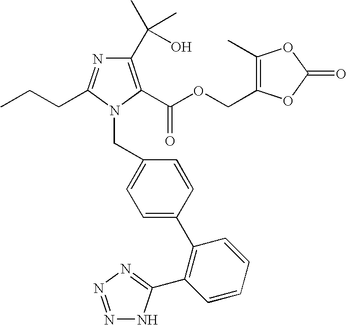

The empirical formula is C₂₉H₃₀N₆O₆.

The molecular weight is 558.58.

Olmesartan medoxomil is a prodrug that is hydrolyzed during absorption, and it is a selective AT₁ subtype angiotensin II receptor antagonist. Olmesartan medoxomil is disclosed by U.S. Pat. No. 5,616,599 to Yanagisawa et al. It is marketed as BENICAR® in film-coated tablets of 5 mg, 20 mg, and 40 mg for treatment of hypertension in a human.

The synthesis of olmesartan medoxomil (OLM-Mod) per se is illustrated in Scheme 1 (see also Annu. Rep. Sankyo Res. Lab 2003, 55, 1-91):

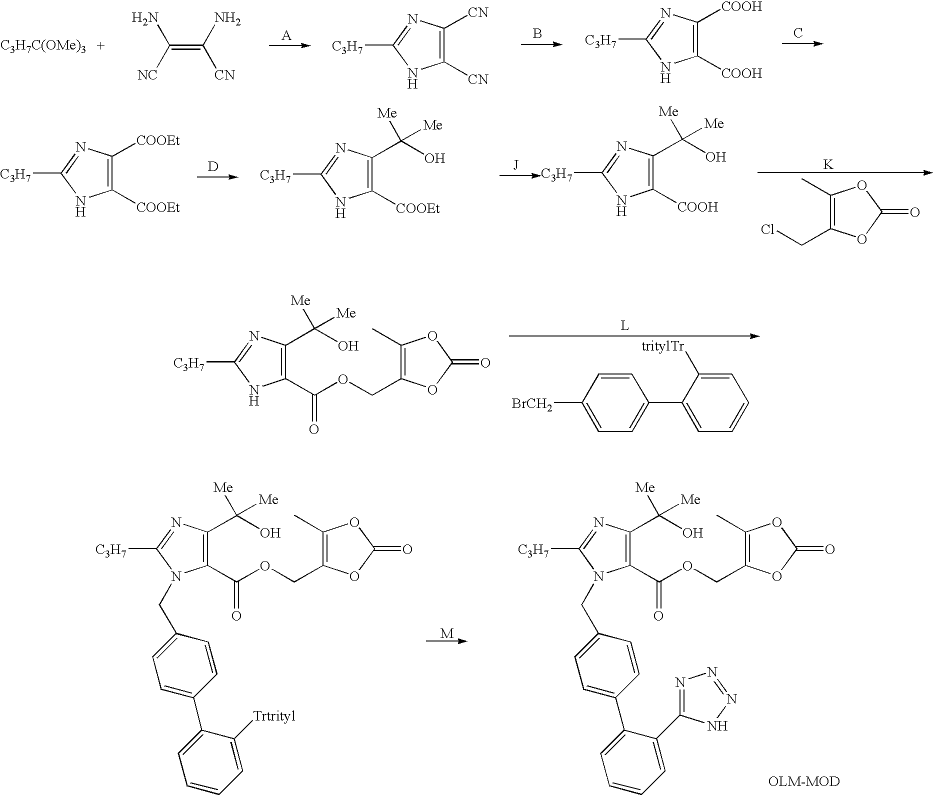

In this scheme, “tritylTr” relates to triphenyl tetrazole.

This route of synthesis produces several impurities.

There is a need for processes for preparing olmesartan medoxomil with reduced levels of impurities.

### BRIEF DESCRIPTION OF THE FIGURES

FIG. 1 depicts a typical chromatogram of a trityl olmesartan medoxomil (MTT) sample.

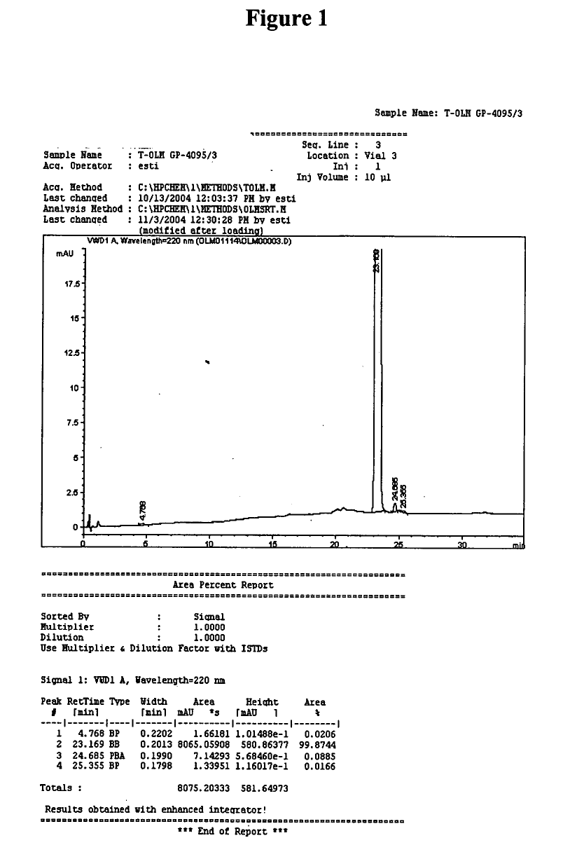

### SUMMARY OF THE INVENTION

In one aspect, the present invention provides processes for preparing olmesartan medoxomil containing less than about 0.1% area by HPLC of one or more of the following impurities: OLM-Me, OLM-Cl, and OLM-eliminate.

A process for preparing olmesartan medoxomil containing less than about 0.1% area by HPLC of one or more of OLM-Me, OLM-Cl, and OLM-eliminate comprises: obtaining a sample of trityl olmesartan medoxomil (MTT); measuring the amount of one or more impurities selected from the group consisting of MTT-Me, MTT-Cl, and MTT-eliminate in the sample of trityl olmesartan medoxomil; selecting a sample of trityl olmesartan medoxomil in which the amount of one or more of the measured impurities is less than about 0.1%; and synthesizing olmesartan medoxomil from the selected trityl olmesartan medoxomil sample. Preferably, the amount of each of the three impurities in the starting material and/or the final product is less than about 0.1%. More preferably, the combined amount of the three impurities is less than about 0.1%.

Another process for preparing olmesartan medoxomil containing less than about 0.1% area by HPLC of one or more of OLM-Me, OLM-Cl, and OLM-eliminate comprises: obtaining a sample of ethyl 4-(1-hydroxy-1methylethyl)-2-propylimidazole-5-carboxylate (defined here as ethyl imidazole 5-carboxylate); measuring the amount of one or more impurities selected from the group consisting of D-Me and D-eliminate in the sample; selecting a sample of ethyl-imidazole 5-carboxylate in which the amount of one or more of the measured impurities is less than about 0.1%; and synthesizing olmesartan medoxomil from the selected sample. Preferably, the amount of each of the two impurities in the starting material and/or the final product is less than about 0.1%. More preferably, the combined amount of the two impurities is less than about 0.1%.

Another process for preparing olmesartan medoxomil containing less than about 0.1% area by HPLC of one or more of OLM-Me, OLM-Cl, and OLM-eliminate comprises: obtaining a sample of (5-methyl-2-oxo-1,3-dioxolen-4-yl)methyl-4-(hydroxyl-1methylethyl)-2-propylimidazole-5-carboxylate (defined here as medoxomil-imidazole 5-carboxylate); measuring the amount of one or more impurities selected from the group consisting of K-Me, K-eliminate and K-Cl in the sample; selecting a sample of medoxomil-imidazole 5-carboxylate in which the amount of one or more of the measured impurities is less than about 0.1%; and synthesizing olmesartan medoxomil from the selected sample. Preferably, the amount of each of the three impurities in the starting material and/or the final product is less than about 0.1%. More preferably, the combined amount of the three impurities is less than about 0.1%.

### DETAILED DESCRIPTION OF THE INVENTION

The present invention provides a process for preparing olmesartan medoxomil containing less than about 0.1% area by HPLC of one or more of the impurities OLM-Me, OLM-Cl and OLM-eliminate.

Impurity OLM-Me is 4-(1-methoxy-1-methylethyl)-2-propyl-1-[2′-(1H-tetrazole-5-yl)biphenyl-4-ylmethyl]imidazole-5-carboxylic acid 5-methyl-2-oxo-1,3-dioxol-4-ylmethyl ester.

Impurity OLM-Cl is 4-(1-hydroxy-1-methylethyl)-2-propyl-1-[2′-(1H-tetrazole-5-yl)biphenyl-4-ylmethyl]imidazole-5-carboxylic acid 5-chloromethyl-2-oxo-1,3-dioxol-4-ylmethyl ester.

Impurity OLM-eliminate is 4-(1-methylethylene)-2-propyl-1-[2′-(1H-tetrazole-5-yl)biphenyl-4-ylmethyl]imidazole-5-carboxylic acid 5-chloromethyl-2-oxo-1,3-dioxol-4-ylmethyl ester.

The chemical structures of impurities OLM-Me, OLM-Cl, and OLM-eliminate are:

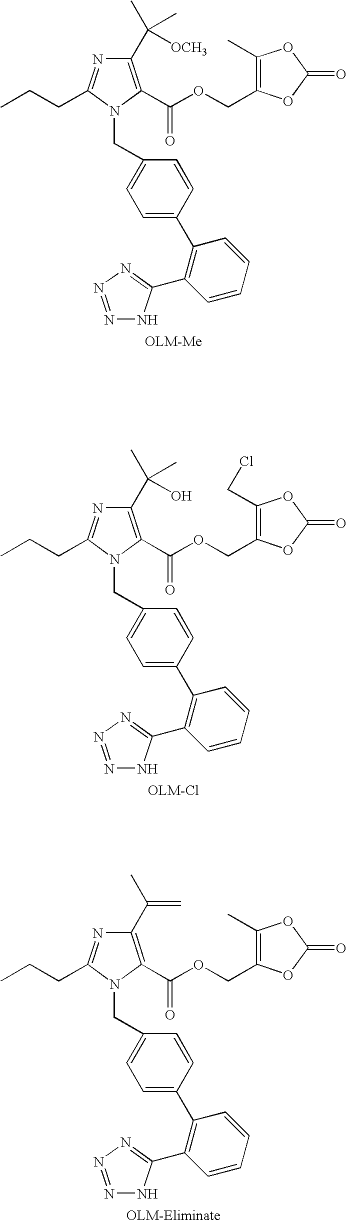

The precursors of impurities OLM-Me and OLM-eliminate can form during the Grignard reaction, in reaction step D, or during reaction step K, in the synthesis route described previously (represented in Scheme I). The formation of the precursors of impurity OLM-Me (denominated D-Me and K-Me) and of impurity OLM-eliminate (denominated D-eliminate and K-eliminate) is illustrated as follows:

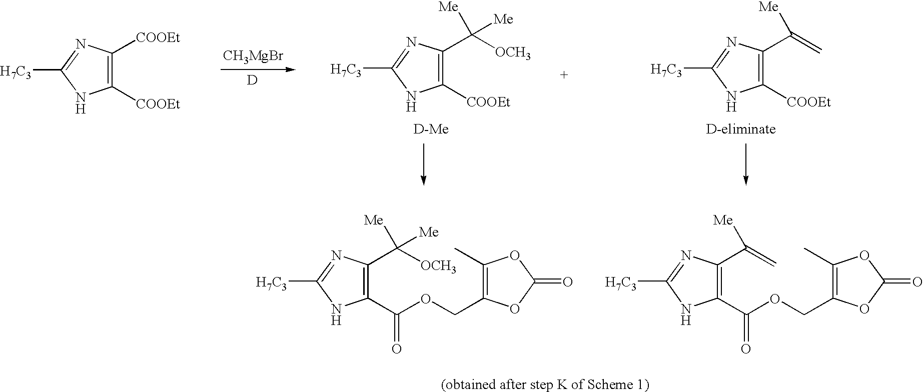

The precursor of impurity OLM-Cl (denominated K-Cl) can form during reaction step K when the coupling reagent chloro-medoxomil (4-chloromethyl-2-oxo-1,3-dioxolene) contains some dichloromedoxomil (4,5-dichloro-dimethyl-2-oxo-1,3-dioxolene):

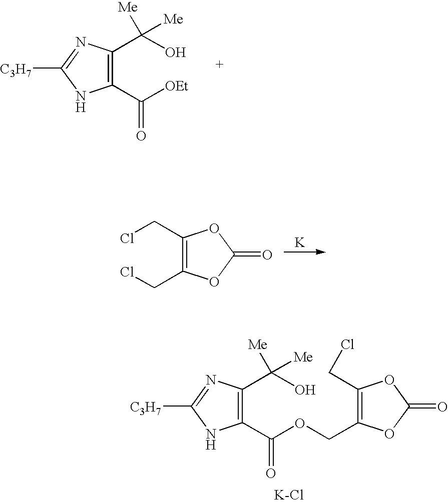

The precursors obtained by the steps shown above can react with a bromomethane derivative (step L in Scheme 1) and undergo hydrolysis (step M in Scheme 1), thus impurities OLM-Me, OLM-Cl and OLM-eliminate are formed.

The impurities OLM-Me, OLM-Cl, and OLM-eliminate or their precursors have no known medicinal effect. The impurities at the trityl olmesartan medoxomil (MTT) stage—MTT-Me, MTT-Cl, and MTT eliminate—are not used for synthesizing olmesartan medoxomil. Structures for MTT-Me, MTT-Cl, and MTT are described below.

By selecting trityl olmesartan medoxomil or a precursor thereof with low levels of their respective impurities (MTT-Me, MTT-Cl, and MTT eliminate for trityl olmesartan medoxomil, D-Me and D-eliminate for when selecting ethyl-imidazole 5-carboxylate_as a starting material, or K-Me, K-eliminate and K-Cl for when selecting medoxomil-imidazole 5-carboxylate as a starting material, one can use the selected starting material to synthesize olmesartan medoxomil with low levels of impurities.

In one embodiment, the present invention provides a process for preparing olmesartan medoxomil containing less than about 0.1% area by HPLC of one or more of the following impurities: OLM-Me, OLM-Cl, and OLM-eliminate. This process comprises: obtaining a sample of trityl olmesartan medoxomil (MTT); measuring the amount of one or more impurities selected from the group consisting of MTT-Me, MTT-Cl, and MTT-eliminate in the sample of trityl olmesartan medoxomil; selecting a sample of trityl olmesartan medoxomil in which the amount of one or more of the measured impurities is less than about 0.1%; and synthesizing olmesartan medoxomil from the selected trityl olmesartan medoxomil sample. Preferably, the amount of each of the three impurities in the starting material and/or the final product is less than about 0.1%. More preferably, the combined amount of the three impurities is less than about 0.1%.

The chemical structures of MTT-Me, MTT-Cl, and MTT-eliminate are:

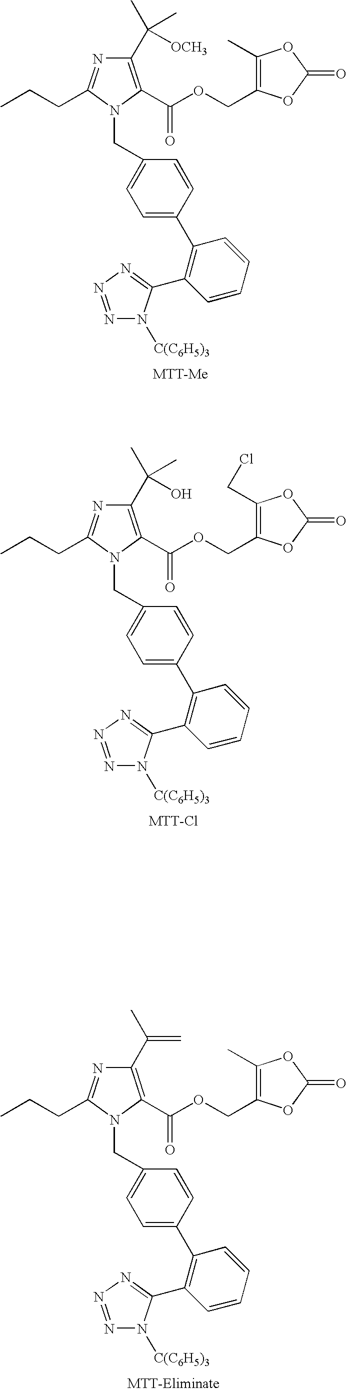

The amounts of MTT-Me, MTT-Cl, and MTT-eliminate are measured using HPLC. The amounts of OLM-Me, OLM-Cl and OLM-eliminate are also measured using HPLC.

In one embodiment, the present invention provides a process for preparing olmesartan medoxomil containing less than about 0.1% area by HPLC of one or more of the following impurities: OLM-Me and OLM-eliminate. This process comprises: obtaining a sample of ethyl-imidazole 5-carboxylate; measuring the amount of one or more impurities selected from the group consisting of D-Me and D-eliminate in the sample of ethyl-imidazole 5-carboxylate; selecting a sample of ethyl-imidazole 5-carboxylate in which the amount of one or more of the measured impurities is less than about 0.1%; and synthesizing olmesartan medoxomil from the selected ethyl-imidazole 5-carboxylate sample. Preferably, the amount of each of the impurities in the starting material and/or the final product is less than about 0.1%. More preferably, the combined amount of the impurities is less than about 0.1%.

In one embodiment, the present invention provides a process for preparing olmesartan medoxomil containing less than about 0.1% area by HPLC of one or the one or more of the following impurities: OLM-Me, OLM-eliminate and OLM-Cl. This process comprises: obtaining a sample of medoxomil-imidazole 5-carboxylate; measuring the amount of one or more impurities selected from the group consisting of K-Me, K-eliminate and K-Cl in the sample of medoxomil-imidazole 5-carboxylate; selecting a sample of medoxomil-imidazole 5-carboxylate in which the amount of the measured impurity is less than about 0.1%; and synthesizing olmesartan medoxomil from the selected medoxomil-imidazole 5-carboxylate sample. Preferably, the amount of the impurity in the starting material and/or the final product is less than about 0.1%.

One can use any method known in the art to synthesize olmesartan medoxomil from trityl olmesartan medoxomil, such as the process described in U.S. Pat. No. 5,616,599. Olmesartan medoxomil can be synthesized from trityl olmesartan medoxomil by a method comprising: contacting trityl olmesartan medoxomil with an acid in a water miscible organic solvent, with or without water, to obtain a solution of olmesartan medoxomil and a precipitate of triphenyl carbinol; separating the precipitate of triphenyl carbinol from the solution of olmesartan medoxomil; and contacting the solution of olmesartan medoxomil with a base to obtain a precipitate of olmesartan medoxomil. Preferably, trityl olmesartan medoxomil is contacted with the acid in a water miscible organic solvent and water. Most preferably, a mixture of acetone and water is used.

### EXAMPLES

### Impurity Profile Determination of MTT (Raw Material of Olmesartan Medoxomil)

HPLC

| Column & packing | Discovery HS C18 50 * 4.6 mm, 3μ C.N 269250-U | | |
|---|---|---|---|
| Eluent A: | 0.025 M NaClO₄ adjusted to pH = 2.5 with HClO₄ | | |
| Eluent B: | Acetonitrile | | |
| Gradient of Eluent: | Time (min) | Eluent A (%) | Eluent B (%) |
| | 0 | 70 | 30 |
| | 10 | 60 | 40 |
| | 20 | 40 | 60 |
| | 35 | 40 | 60 |

| | |
|---|---|
| Stop time: | 35 min |
| Equilibration time: | 5 min |
| Flow: | 1.5 ml/min |
| Detector: | 220 nm |
| Injection volume: | 10 μl |
| Diluent | Acetonitrile |
| Column temperature | 25° C. |
| Autosampler | 5° C. |
| temperature | |

Sample Solution Preparation

Weigh accurately about 15 mg of MTT sample into a 50 ml volumetric flask, dissolve, and dilute to volume with diluent.

Method

Inject sample solutions continuing the chromatogram up to the end of gradient.

Determine the area of each impurity using suitable integrator.

Calculations

Any impurity in a sample is calculated as follows:

$$\% Impurity in sample = \frac{area impurity in sample}{\sum Areas of all peaks} \times 100$$

RRT of the Substances

| Substance | RT | RRT |
|---|---|---|
| TPC | 16.28 | 0.70 |
| MTT | 23.20 | 1.00 |
| MTT-Me | 24.70 | 1.06 |
| MTT-Cl | 24.96 | 1.08 |
| MTT-Eliminate | 25.33 | 1.09 |

The detection limit in the HPLC method is 0.01%.

### Example 1

### Preparation of Crude Olmesartan Medoxomil

A 250 round bottom flask was loaded with MTT (10 g), acetone/water (2/2 vol.), and 3 eq of H₂SO₄. The mixture was stirred at 40° C., and after 2-4 hrs, triphenyl carbinol (TPC) was precipitated by the addition of water and filtrated out. NaHCO₃ was added to the filtrate and the mixture was cooled to room temperature and stirred for 1 hr. Crude olmesartan medoxomil was obtained as white crystals (90% yield).

### Example 2

### Preparation of Olmesartan Medoxomil Crystals

A 1 L flask was charged with acetone (4% water). Crude olmesartan medoxomil (10 g) was added, and the mixture was heated to reflux (1 hr). After cooling to room temperature, water (10 vol) was added. The mixture was stirred (1 hr). Then the precipitate was filtered and dried at 45° C. under 10 mm Hg (yield 90%).

Having thus described the invention with reference to particular preferred embodiments and illustrative examples, those in the art can appreciate modifications to the invention as described and illustrated that do not depart from the spirit and scope of the invention as disclosed in the specification. The examples are set forth to aid in understanding the invention but are not intended to, and should not be construed to, limit its scope in any way. The examples do not include detailed descriptions of conventional methods.

What is claimed is:

1. A process for preparing olmesartan medoxomil containing less than about 0.1% of one or more of OLM-Me, OLM-Cl, and OLM-eliminate, comprising:
a) obtaining a sample of trityl olmesartan medoxomil;
b) measuring the amount of one or more impurities selected from the group consisting of MTT-Me, MTT-Cl, and MTT-eliminate in the sample of trityl olmesartan medoxomil;
c) selecting a sample of trityl olmesartan medoxomil in which the amount of one or more of the measured impurities is less than about 0.1%; and
d) synthesizing olmesartan medoxomil from the trityl olmesartan medoxomil selected in step c).

2. The process of claim 1, wherein the amount of each of the impurities MTT-Me, MTT-Cl, and MTT-eliminate in the selected sample of step c) is less than about 0.1%.

3. The process of claim 2, wherein the amount of MTT-Me in the selected sample of step c) is less than about 0.1%.

4. The process of claim 1, wherein the combined amount of the impurities MTT-Me, MTT-Cl, and MTT-eliminate in the selected sample of step c) is less than about 0.1%.

5. The process of claim 1, wherein the one or more impurities in step b) is measured by HPLC.

6. The process of claim 1, wherein the amount of each of the impurities OLM-Me, OLM-Cl, and OLM-eliminate in the olmesartan medoxomil synthesized in step d) is less than about 0.1%.

7. The process of claim 6, wherein the amount of OLM-Me in the olmesartan medoxomil synthesized in step d) is less than about 0.1%.

8. The process of claim 1, wherein the combined amount of the impurities OLM-Me, OLM-Cl, and OLM-eliminate in the olmesartan medoxomil synthesized in step d) is less than about 0.1%.

9. The process of claim 1, wherein the impurities OLM-Me, OLM-Cl, and OLM-eliminate in the olmesartan medoxomil synthesized in step d) are measured by HPLC.

10. A process for preparing olmesartan medoxomil containing less than about 0.1% of one or more of OLM-Me and OLM-eliminate, comprising:
a) obtaining a sample of ethyl- imidazole 5-carboxylate;
b) measuring the amount of one or more impurities selected from the group consisting of D-Me and D-eliminate in the sample of ethyl-imidazole 5-carboxylate;
c) selecting a sample of ethyl-imidazole 5-carboxylate in which the amount of one or more of the measured impurities is less than about 0.1%; and
d) synthesizing olmesartan medoxomil from the ethyl-imidazole 5-carboxylate selected in step c).

11. The process of claim 10, wherein the amount of each of the impurities D-Me and D-eliminate in the selected sample of step c) is less than about 0.1%.

12. The process of claim 10, wherein the combined amount of the impurities D-Me and D-eliminate in the selected sample of step c) is less than about 0.1%.

13. The process of claim 10, wherein the one or more impurities in step b) is measured by HPLC.

14. The process of claim 10, wherein the amount of each of the impurities OLM-Me and OLM-eliminate in the olmesartan medoxomil synthesized in step d) is less than about 0.1%.

15. The process of claim 14, wherein the amount of OLM-Me in the olmesartan medoxomil synthesized in step d) is less than about 0.1%.

16. The process of claim 10, wherein the combined amount of the impurities OLM-Me and OLM-eliminate in the olmesartan medoxomil synthesized in step d) is less than about 0.1%.

17. The process of claim 10, wherein the impurities OLM-Me and OLM-eliminate in the olmesartan medoxomil synthesized in step d) are measured by HPLC.

18. A process for preparing olmesartan medoxomil containing less than about 0.1% of one or more of OLM-Me, OLM-eliminate and OLM-Cl, comprising:
a) obtaining a sample of medoxomil-imidazole 5-carboxylate;
b) measuring the amount of one or more impurities selected from the group consisting of K-Me, K-eliminate and K-Cl in the sample of medoxomil-imidazole 5-carboxylate;
c) selecting a sample of medoxomil-imidazole 5-carboxylate in which the amount of the measured impurities is less than about 0.1%; and
d) synthesizing olmesartan medoxomil from the medoxomil-imidazole 5-carboxylate selected in step c).

19. The process of claim 18, wherein the amount of each of the impurities K-Me, K-eliminate and K-Cl in the selected sample of step c) is less than about 0.1%.

20. The process of claim 18, wherein the combined amount of the impurities K-Me, K-eliminate and K-Cl in the selected sample of step c) is less than about 0.1%.

21. The process of claim 18, wherein the impurity in step b) is measured by HPLC.

22. The process of claim 18, wherein the amount of the impurities OLM-Me, OLM-eliminate and OLM-Cl in the olmesartan medoxomil synthesized in step d) is less than about 0.1%.

23. The process of claim 22, wherein the amount of OLM-Me in the olmesartan medoxomil synthesized in step d) is less than about 0.1%.

24. The process of claim 18, wherein the combined amount of the impurities OLM-Me, OLM-eliminate and OLM-Cl in the olmesartan medoxomil synthesized in step d) is less than about 0.1%.

25. The process of claim 18, wherein the impurities OLM-Me, OLM-eliminate and OLM-Cl in the olmesartan medoxomil synthesized in step d) is measured by HPLC.
